(19) United States
(12) Patent Application Publication
(10) Pub. No.: US 2025/0297974 A1
(43) Pub. Date: Sep. 25, 2025

(54) DETECTING TARGET MOLECULES USING ALPHA PARTICLE RADIOISOTOPES

(71) Applicants: THE EARTHINEERING COMPANY LLC, Claremont, CA (US); Keck Graduate Institute of Applied Life Sciences, Claremont, CA (US)

(72) Inventors: Kevin Peter Hickerson, Claremont, CA (US); Travis Schlappi, Mesa, AZ (US)

(73) Assignees: The Earthineering Company, Wilmington, DE (US); Keck Graduate Institute of Applied Life Sciences, Claremont, CA (US)

(21) Appl. No.: 18/864,204

(22) PCT Filed: Mar. 13, 2023
(86) PCT No.: PCT/US2023/064229

§ 371 Date: Nov. 8, 2024

Related U.S. Application Data
(60) Provisional application No. 63/341,726, filed on May 13, 2022.

(51) Int. Cl. G01N 23/22; G01N 33/487; G01N 33/543; G01N 33/569

(52) U.S. Cl. CPC G01N 23/22; G01N 33/487; G01N 33/54388; G01N 33/56983

(57) ABSTRACT

A diagnostic device and method detect target molecules in a liquid sample using radioisotopes that undergo alpha decay. The liquid sample is introduced onto a first portion of a permeable membrane, where its target molecules chemically bind to molecular tags. Each molecular tag includes both an alpha-emitter and a capture molecule designed to bind to the target molecule. The sample, including any bound target molecules, undergoes capillary flow to a second portion of the membrane, where an alpha-particle detector detects any bound alpha-emitters. The alpha-emitters may be radioactive nanoparticles (e.g. polonium-210) coated with an environmental protectant (e.g. gold). The detector may include CMOS diodes or a charge-coupled device. The device may include an indicator that signals when the detector detects alpha-particles above a given threshold in the second portion.

### GOVERNMENT LICENSE RIGHTS

This invention was made with government support under R03AI169303 from the National Institute of Allergy and Infectious Diseases. The government has certain rights in the invention.

### FIELD

The disclosure pertains generally to apparatus for diagnosis and detection of target molecules using radiation, and more particularly to investigating or analyzing biomaterials by the use of alpha (α) particle radiation.

### BACKGROUND

Diagnosis of infectious diseases is ineffective when the diagnostic test does not meet one or more of the necessary standards of affordability, accessibility, and accuracy. The World Health Organization further clarifies with the acronym ASSURED: Affordable, Sensitive, Specific, User-friendly, Rapid and robust, Equipment-free or simple, and Deliverable to end-users.

The shortcomings of current diagnostic methods have been apparent in the COVID-19 pandemic, where some tests are accurate, but not affordable or accessible (e.g. RT-PCR tests that detect COVID RNA), while other tests have become more accessible and affordable, but have low accuracy (e.g. rapid antigen tests). In the early stages of the pandemic, the delay for receiving PCR results was very long (multiple days and sometimes 1-2 weeks). In the later stages of the pandemic, rapid diagnostic tests (RDT) that detect COVID antigens became available at a cost of $5-10 with a turnaround time of 15 minutes, but studies showed that they detected only 53.3% of asymptomatic carriers compared to the RT-PCR assay. The high false negative rate of rapid antigen tests precludes public health officials' ability to limit disease transmission; therefore, RT-PCR or other nucleic acid (NA) tests remain the preferred testing method.

These tradeoffs of good sensitivity with high cost and slow turnaround time, or poor sensitivity with low cost and fast turnaround time, have only been problems in the US and other high-income countries (HIC). Low-to-middle income countries (LMIC) struggle to afford either kind of test.

This problematic tradeoff of affordable-but-inaccurate or costly-but-accurate is important not just for COVID-19 and respiratory pandemics, but for other infectious diseases as well, such as human immunodeficiency virus (HIV), Hepatitis C, diarrheal diseases, malaria, antibiotic resistant bacteria, and sexually transmitted infections (STIs). According to one estimate, a test for HIV with 90% sensitivity, 90% specificity and minimal laboratory infrastructure requirements could save up to 2.5 million DALYs (disability-adjusted life years), while a malaria test with 95% sensitivity, 95% specificity, and no laboratory infrastructure requirements could save ˜2.2 million adjusted lives and prevent ˜447 million unnecessary treatments per year. The reason that accurate tests (NA tests, enzyme immunoassays, and others) need laboratory infrastructure and are only available in Levels 2-4 of FIG. 1 is because they rely on complex biochemical methods that require sophisticated instrumentation to perform multiple steps—adding reagents, mixing, washing, incubating at specific temperatures, and detecting fluorescence. An affordable (<$1 per test), accessible (Levels 0-1 of FIG. 1), and accurate (>90% sensitivity and specificity) test is needed to improve infectious disease detection, management, and treatment selection for both HICs and LMICs.

Having access to a rapid, affordable, and accurate diagnostic test would make drastic improvements to the management of a variety of infectious diseases. For example, a rapid and inexpensive COVID test could help asymptomatic people with COVID infection know that they are infected and quarantine; a rapid and inexpensive malaria test could differentiate malaria infections from other causes of fever, stop overtreatment of anti-malaria drugs, and slow the spread of multi-drug resistant malarial infections; a rapid and inexpensive urinary tract infection or STI test could help patients receive the correct antibiotic in their first physician visit, thus curbing the spread of antibiotic resistance, superbugs, and preventing the more serious complications that resistant infections cause.

Much research has been done to improve current methods of detecting pathogenic organisms, nucleic acids (NAs), and indicative proteins to make them less expensive, accessible to the community, and more accurate. However, the critical barrier to making progress is that the bacteria, viruses, NAs, or proteins of interest exist in the respiratory, blood, diarrheal, or urine sample in too low of a concentration to be directly detected. For example, COVID antigens in asymptomatic carriers can range from 0.01 to 5000 μg/mL in saliva and nasopharyngeal swabs, with 50% of the samples being <4.00 μg/mL. Rapid antigen tests have LODs ranging from 3 to 22 μg/mL, meaning that even the best rapid antigen tests would miss approximately half of asymptomatic carriers. These comparisons agree with the study mentioned earlier that showed a 53.3% sensitivity compared to RT-PCR for the BinaxNOW COVID-19 antigen test. To achieve the sensitivities of >90%, current methods therefore amplify the pathogenic organism (e.g. culturing bacteria or viruses), amplify a target biomolecule coming from the pathogen (e.g. polymerase chain reaction (PCR) for NAs), or amplify a signal indicating the presence of a pathogenic organism (e.g. enzyme-linked immunosorbent assay (ELISA) for proteins). Even with recent advances, these amplification methods still require many steps and costly instruments to purify the target molecule from the sample and perform amplification. Avoiding amplification is also an option (e.g. lateral flow RDTs), but sensitivity, accuracy, and utility decrease.

### SUMMARY OF DISCLOSED EMBODIMENTS

Disclosed embodiments use a technology that is inexpensive, rapid, and extremely sensitive—detection of nuclear radiation. Single radioactive emission events can be detected with a portable device, without requiring specific temperatures or reagents found in biological and chemical methods. Radioactive nanoparticles (rNPs) are used as tags for detecting biomolecules. The principle is similar to a lateral flow sandwich immunoassay, wherein gold nanoparticles (NPs) bind to a target protein, localize to a region in a lateral flow strip, and are then detected optically. In our approach, rNPs bind to a target biomolecule, localize to a specific region, and then radioactive emissions (or the lack thereof) are detected with a simple and inexpensive device, such as a complementary metal-oxide semiconductor (CMOS) sensor. Because single emission events from the rNP can be detected, far fewer target molecules are needed to localize in a region to emit a detectable signal, and the sensitivity is significantly increased. Clinical practice, disease management, pandemic preparedness, and healthcare of citizens around the globe would be transformed with rapid, accurate, accessible, and affordable tests for infectious diseases. While we are aware of concerns with using nuclear technology, we believe that the benefits of this diagnostic technology to schools, transportation, bioterrorism defense, and society, outweigh the potential risks (similar to how the benefits of a smoke detector outweigh the perceived risk of nuclear radiation from its americium source). Moreover, we have implemented mitigation strategies and safety measure to make this diagnostic test no more risky than having smoke detectors in one's home.

### DESCRIPTION OF THE SEVERAL VIEWS OF THE DRAWINGS

The manner and process of making and using the disclosed embodiments may be appreciated by reference to the drawings, in which:

FIGS. 1(a) and 1(b) show test tradeoffs at different levels of the health care system;

FIG. 2 shows a schematic of a test device for detecting the presence of a target molecule in a liquid sample using a radiological assay in accordance with a first embodiment of the concepts, techniques, and structures disclosed herein;

FIG. 2A is a flowchart of an assay for detecting the presence of a target molecule in a liquid sample in accordance with a second embodiment;

FIG. 3 shows the dependence of detectable α decay (measured in Becquerels on the left axis) and device radiation (measured in micro-Curies on the right axis) on emitter half-life for various nanoparticle diameters;

FIGS. 4(a) to 4(c) show various modifications of a CMOS sensor under a radiation;

FIG. 5 shows a test apparatus for measuring dead layer, back scatter, and energy emitted by the sealed source of FIG. 4;

FIG. 6 shows an exterior view of a lateral flow device in accordance with a third embodiment of the concepts, techniques, and structures disclosed herein;

FIG. 7 shows an exploded view of the lateral flow device of FIG. 6;

FIG. 8 shows a point-of-care (POC) device with cartridge inserted, in accordance with a fourth embodiment of the concepts, techniques, and structures disclosed herein;

FIG. 9 shows an exploded view of a cartridge suitable for insertion in the POC device of FIG. 8; and

FIG. 10 shows a top view of the cartridge of FIG. 9 with relevant components labeled.

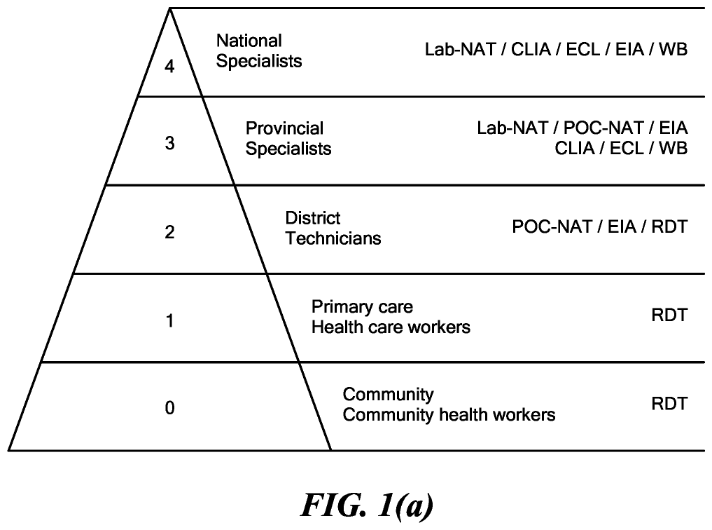

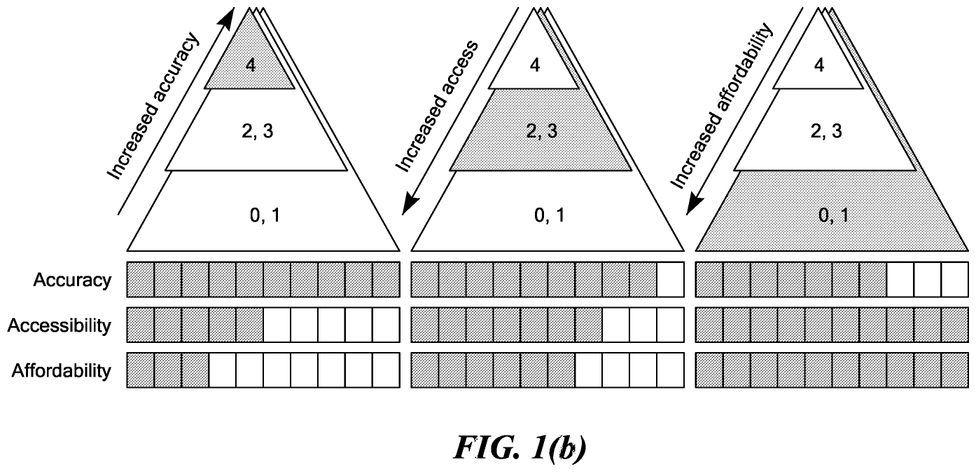

### DETAILED DESCRIPTION OF EMBODIMENTS

As used herein, “nanoparticle” means a particle having a diameter that is at most 1 millimeter.

Current clinical practice includes diagnostic tests if they are accessible and affordable, which means that most LMICs do not use diagnostics and are set up to make incorrect treatment decisions and spread disease unknowingly. Even in HICs, screening large populations for COVID-19 is not recommended due to the cost and inadequate sensitivity of RDTs. With an inexpensive, accessible, and accurate testing method, clinicians and patients alike will be able to test for disease far more often and effectively, thus providing the information necessary to stop disease spread and effectively treat those who have been infected.

For stopping disease spread, a diagnostic test must prioritize sensitivity (minimizing false negatives) at the expense of specificity (minimizing false positives). Unfortunately, due to the chemical detection methods used in current RDTs, specificity is great (>99% in for many RDTs) but sensitivity suffers, particularly for asymptomatic carriers. Due to the ultra-high sensitivity of alpha (α) particle detection, our diagnostic device naturally biases towards false positives rather than false negatives, which will result in a small percentage of healthy people unnecessarily quarantining, but with the more important benefit of catching asymptomatic or pre-symptomatic carriers before they unknowingly spread the disease. This is similar to smoke detectors, which rely on alpha particle detection from americium-241 and have false positive issues when other particulate dust is mistaken for smoke. However, users and regulators of smoke detectors have accepted this false positive risk and alpha radiation emitters in their homes given the benefits of high sensitivity fire detection (via smoke interacting with the alpha particles).

Radioisotopes have been routinely used in medicine for decades to image blood flow, specific organs, or treat cancer. The most widely used radioisotope for in vivo imaging is technetium-99, which emits gamma (γ) rays through the body that can be detected by an external γ camera. It has a half-life of 6 hours and accounts for 40 million procedures per year. Iodine-131 is a radionuclide taken up by the thyroid, which makes it a common and relatively successful treatment for thyroid cancer. While these radioisotopes are effective for their purposes of organ imaging and cancer treatment, they have a short half-life and thus require the hospital to have a hot reactor constantly making the radioisotope. To develop an affordable, accessible and accurate diagnostic device for infectious disease, we identified three key innovations to current nuclear medicine practice: i) detecting α emissions instead of γ rays, ii) mixing radioisotopes with in vitro samples rather than infusing into a patient in vivo, and iii) using radioisotopes with half-lives of several years or more, thus eliminating the need for a hot reactor and enabling the diagnostic test to be shelf stable and portable to remote communities.

Detecting α emissions instead of gamma (γ) or beta (β) rays is beneficial for several reasons: α particles have very large energies per particle, have a short range due to their large mass, and correctly packaged alphas pose no risk of harm to the user. Alphas have energies in the range of 3-8 megaelectron volts (MeV), whereas gammas are typically only 0.5-2 MeV. Further, because alphas are quite massive, 3.727 GeV, just under the mass of a helium atom, and thousands of times more mass than a beta particle, they deposit their energy in very short distances as predicted by the Bragg curve. This makes them much easier to detect, because they create many ions in a small amount of material. This also makes them much safer to handle outside the human body, because their short stopping distance makes them unable to penetrate even a small amount of shielding, such as paint, plastic or a piece of paper. Thus, even a small, low voltage detector can spot them, such as photodiodes in smartphone image sensors.

Single alphas can be counted and their location on the sensor can be measured to within a few tens of microns. On modern sensors, this is only a few pixels. This not only makes detecting the α possible, but several different analytes can be tested at once as a grid across the sensor. γ detectors by contrast, require large single crystal volumes and high voltage amplification systems. These devices are large, expensive, delicate, and are rarely used in medicine outside of a large hospital imaging system such as a PET scanner. There is also much more background γ radiation than α, so distinguishing a γ ray from background would require much stronger sources than we ever hope to require. Alphas deposit their energy in such a small space that their tracks can be imaged, and their energy measured with reasonable accuracy to separate their detection from background events and electronic noise.

In vitro detection of bodily fluids (urine, feces, blood, saliva, etc.) ensures that the procedure is non-invasive and does not require trained personnel, which has been a major challenge for other diagnostic devices. The short stopping distance of alphas also enables the radioactive material to be well contained within the device, protected by an x-ray blocking plastic seal, and pose no to minimal risk of radioactive exposure.

Embodiments use radioisotopes in an inexpensive diagnostic device with a long (months to years) shelf-life and no risk to the patient or healthcare workers administering the test. If successful, the sensitivity, simplicity, and low cost of radiological detection will enable us to meet all ASSURED criteria for effective diagnostic tests. Our goal for this proposed work is to develop a device that has the simplicity and low cost of a lateral flow test, but the sensitivity of a PCR test.

It is important to emphasize the safety of both the proposed device and the research we will do to investigate this hypothesis. Simply put, an α-emitting source poses no danger to a user as long as the source isotopes are kept inside its packaging and not ingested or inhaled. The large mass of α particles means that virtually all the energy emitted from a radioactive decay is deposited in just a few tens of microns of shielding (plastic, paint or paper). No thick and heavy lead is required. Very low-cost shielding such as a foil of plastic cartridge will be all that is needed to protect experimenters, patients, doctors, and the environment from any exposure to harmful radiation.

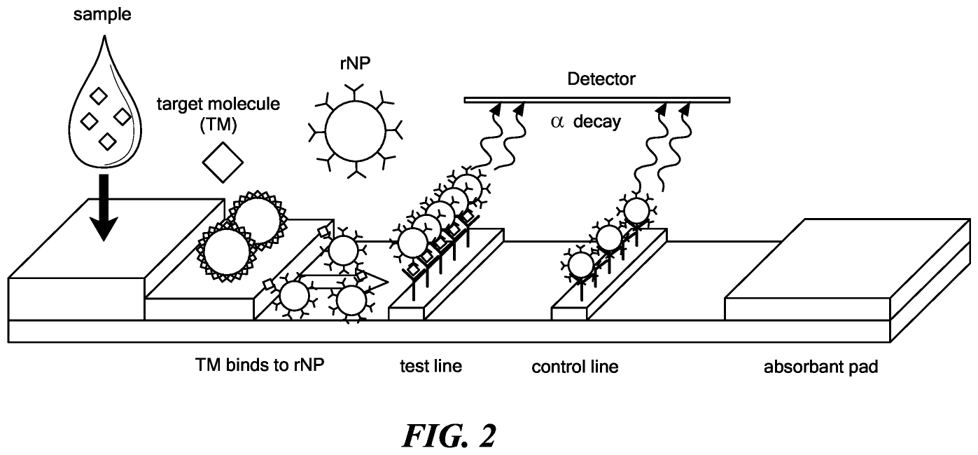

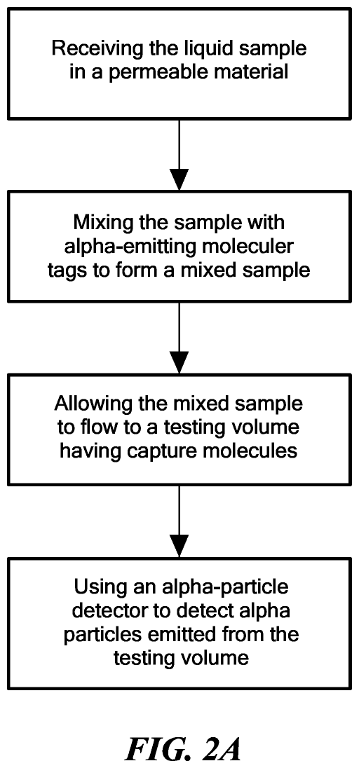

FIG. 2 shows a test device for detecting the presence of a target molecule (TM) in a liquid sample using a radiological assay in accordance with a first embodiment of the concepts, techniques, and structures disclosed herein. This test device uses radioisotopes to detect biomolecules with high sensitivity and low cost. We developed a prototype using simple biomolecules such as human chorionic gonadotropin (hCG), and anti-hCG antibodies (Abs) before testing our methods with COVID-19 nucleocapsid protein antigens. The nanoparticles (NPs) were initially made from naturally abundant elements to develop formulation procedures and Ab functionalization, after which NPs were doped with α-emitting radioisotopes.

In this disclosure, the target molecule comprises a pathogen, or an antigen, or deoxyribonucleic acid (DNA), or ribonucleic acid (RNA), or a toxin. However, it is appreciated that the target molecule need not be organic, provided that suitable molecular tags and capture molecules can be fabricated to bind to the target molecule (or target atom, as the case may be). Therefore, while the remainder of this disclosure frames embodiments in terms of biomolecules, target molecules and embodiments for their detection need not be so limited.

The test device of FIG. 2 is similar to standard sandwich immunoassays. It has a permeable material defining at least a first portion and a second portion, the portions being in the same plane so as to permit capillary flow communication with each other.

The first portion receives the liquid sample, and includes a plurality of molecular tags. Each molecular tag has an α-emitter coupled to capture molecules for binding to the target molecule in its presence. The α-emitter may be a radioactive nanoparticle (rNP), which may be coated with an environmental protectant such as gold. In FIG. 2 the molecular tags are identified as radioactive nanoparticles (rNP), but it is appreciated that embodiments may use other molecular tags in accordance with the concepts, techniques, and structures disclosed herein.

The second portion is used for detecting presence of the target molecule. The second portion includes a testing volume or line having capture molecules for binding to the target molecule in its presence. Preferentially, these capture molecules are the same as the capture molecules used by the molecular tags, but it is appreciated that other capture molecules may be used (e.g. to avoid competing for binding sites on the target molecule).

The test device further has an α-particle detector for detecting α particles emitted from the testing volume. In various embodiments, the α-particle detector includes an array of complementary metal-oxide-semiconductor (CMOS) diodes or a charge-coupled device (CCD).

In some embodiments, the test device has an indicator that indicates when the α-particle detector has detected α particles emitted from the testing volume. The indicator may be visual, audible, or otherwise perceptible, and may use displays or speakers as known in the art. Alternately, the indicator may be electronic and provide a coded signal to an external device (such as a computer) to indicate whether α-particle detection has occurred.

The second portion optionally includes a control volume or line for indicating successful operation of the assay. Where a control volume is used, the first portion of the permeable material may include second molecular tags for binding to non-target molecules known to be present in the liquid sample, the control volume includes second capture molecules for binding to the non-target molecules, and the test device further includes a second detector for detecting the presence of second molecular tags within the control volume.

If the testing volume and the control volume are sufficiently close to each other, and if both molecular tags include α-emitters, then a single α-detector may be used to image emissions from both volumes, as shown in FIG. 2. It is appreciated, however, that if different molecular tags are used and only one is an α-emitter, then two separate detectors may be required (e.g. an α-detector and a visual light detector).

The test device finally includes an absorbent pad or wick as known in the art to absorb the chemical waste produced during the assay.

FIG. 2A is a flowchart of an assay for detecting the presence of a target molecule in a liquid sample according to a second embodiment. The assay shown in FIG. 2A may be performed by the test device shown in FIG. 2, or by other suitable means. A first step of the assay is receiving the liquid sample in a permeable material. The second step is mixing the received liquid sample with a plurality of molecular tags to form a mixed sample, each molecular tag comprising an α-emitter coupled to capture molecules for binding to the target molecule in its presence. The third step is allowing the mixed sample to flow to a testing volume in the permeable material, the testing volume having capture molecules for binding to the target molecule in its presence. And the fourth step is using an α-particle detector to detect α particles emitted from the testing volume.

The remainder of this disclosure discusses ramifications of the above concepts, techniques, and structures during the course of developing and testing a prototype to implement the above-described test device and assay.

The first step in developing the prototype was to select a suitable α-emitting isotope that can be formulated into a nanoparticle (NP). While there are hundreds of different known α-emitting isotopes, we eliminated all isotopes with a half-life less than 1 day from consideration, as shelf stability and portability are essential features of inexpensive and accessible diagnostics. LMIC and remote settings cannot maintain a hot reactor near the testing site to provide rNPs for the manufacture or use of embodiments having a shelf life on the order of only days or weeks. On the other hand, the half-life cannot be too long as the test (and in some embodiments, control) lines require frequent α-decay (>1 decay per second (Becquerel, Bq)) in order for a result to be detected and reported rapidly. The last consideration was radiological safety, and we imposed a maximum of 1 μCurie (μC) on our device, which is what the Nuclear Regulatory Commission (NRC) has deemed safe for smoke detectors (NRC § 30.15 Part 7).

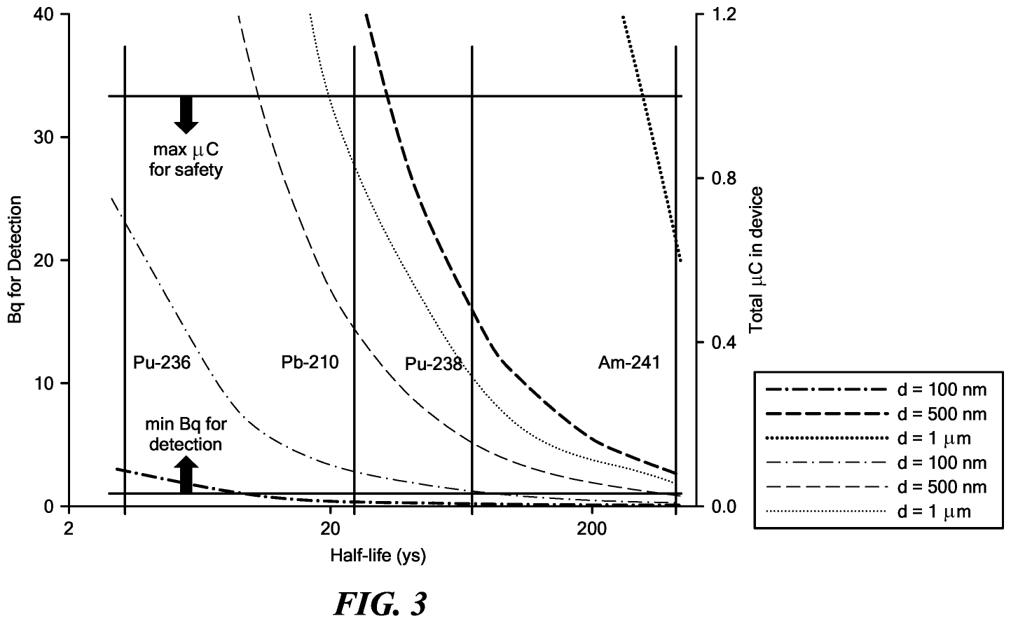

FIG. 3 visualizes this tradeoff, assuming 10% enrichment of the radioisotope in the NP, typical values for antibody surface concentration on α particle (1.4×10⁴ Ab/μm²) and antibody-antigen binding kinetics. We evaluated 4 radioisotopes chosen for their half-life and ease of synthesis or purchase: plutonium-236 (²³⁶Pu), lead-210 (²¹⁰Pb), plutonium-238 (²²⁸Pu), and americium-241 (²⁴¹Am), shown as vertical lines in FIG. 3. It is appreciated that other radioisotopes may be used to emit detectable α radiation in accordance with the concepts, techniques, and structures disclosed herein.

With a half-life of 2.9 years, Pu-236 needs a NP diameter of ˜100 nm in order to have >1 detectable Bqs and <1 μC in the device (NPs with diameter >500 nm result in >1 μC in the device, which could be considered unsafe); Pb-210 and Pu-238 would satisfy both requirements with 500 nm diameter NPs; Am-241 formulated in either 1 μm or 500 nm NPs would satisfy both requirements. It is appreciated that a similar analysis may be used to determine other elements that have a half-life >1 day, 1 week, or 1 year and the optimal NP size.

The naturally abundant element for Am-241 is Am; however europium (Eu) is a suitable surrogate and Eu NPs can be fabricated with a microfluidic co-sol-gel process. The naturally abundant element of the α-emitting Pb-210 is Pb, which is readily available and can be formulated into NPs by treating lead acetate with Cocos nucifera coir extracts. Pu-238 and Pu-236 have a good surrogate in cerium (Ce), and Ce NPs can also be fabricated with a co-sol-gel process. Pu-238 is of particular interest because it is easy to purchase and commercially available, as it is typically used to power spacecraft.

In subsequent work, we recognized that test devices are unlikely to need a shelf life of many years due to chemical decay of other components of the devices. Therefore, we focused our later work on isotopes with a half-life that is less than 2 years, and preferably close to six months.

We also recognized additional considerations in the determination of the radioisotope to use in a commercial device. The first of these was decay branching. As is known in the art, radioisotopes decay into daughter particles, which themselves may be radioactive. Thus, decay chains may be formed, connecting the initial radioisotope with all of its descendant decay products. When a single radioisotope may decay in a number of different ways (e.g. via some combination of α, β, or γ decay), then the chain branches.

Radioisotopes for use in embodiments of the concepts, techniques, and structures disclosed herein preferably have decay chains that include several α decays, as such decay chains emit multiple α particles for each initial radioisotope, multiplying the effectiveness of the α-particle detector. Likewise, effective radioisotopes preferably have a low branching ratio (e.g. between κ% and 10%) with respect to β and γ decays, to increase the chances that the particle emitted from the radioisotope is an α-particle detectable by the α-particle detector.

Moreover, β radiation and internal conversions with an energy above about 1 MeV should be avoided, because high-energy electrons can escape the (e.g. plastic) shielding on a commercial embodiment and pose a minor health risk to tissue outside the testing device, including skin and eyes. For the same reasons, γ (x-ray) radiation above about 50 keV should be avoided.

Decay products in the decay chain (daughter nuclei) should satisfy the above requirements as well. In particular, decay products should also avoid high-energy β and γ radiation that could escape the device shielding. In some cases, the initial radioisotope decays immediately to a stable isotope as the most likely branch (e.g. with a probability of greater than 90%). And the radioisotope preferably does not decay to a radioactive gas, because the gas may not be trappable inside the test device and may be released into the atmosphere. If such a gas cannot be avoided, its release outside the device may be mitigated by chemical bonding to the interior of the test strip.

We have recognized that α particularly useful radioisotope, that satisfies many of the above preferred criteria, is polonium-210 (²¹⁰Po). In particular, polonium-210 has a half-life of 138.4 days, and undergoes α decay to lead-206 (²⁰⁶Pb), which is stable.

There are various tradeoffs in determining which NPs have the best potential to be used as a radioactive signal for the target molecule to be detected. For biomolecule detection, we evaluated the NPs based on the following four criteria.

Criterion 1: How Well can Antibodies be Functionalized onto the Surface of the NPs?

To functionalize antibodies (Abs) onto the surface of metallic NPs, we pursued the induction of amino groups on the surface via treatment with (3-Aminopropyl)triethoxysilane (APTES) via wet chemical procedures. APTES treatment has been shown to be successful at depositing on a variety of solid materials, nanomaterials, and nanocomposites under variable conditions of concentration, solvent, and temperature. Abs were then attached to the amino groups on the surface via standard heterobifunctional cross-linking using 1-Ethyl-3-(3-dimethylaminopropyl) carbodiimide (EDC) and N-hydroxysuccinimide (NHS)/sulfo-NHS; this process forms an amide bond between the amine-functionalized NP and the carboxyl group on the Ab. To determine how well Abs attach to the NP surface, we utilized fluorescence labeling of surface species and fluorescence correlation spectroscopy to measure the amount of Abs that were attached to NPs.

Criterion 2: How Well can Gold (Au) be Coated onto the Surface of the NPs?

Coating the NP surface with Au (or other material to shield the NP from chemically reacting with its environment) will help with both the biochemistry of our detection method and the nuclear physics. Au NPs are commonly used in medical diagnostics and lateral flow strips, with several established methods for attaching antibodies, peptides, oligonucleotides, or polyethylene glycol to the surface. To coat the NPs with Au, we drew from methods in literature, such as sputter coating gold onto cerium oxide NPs, and using sodium citrate reduction of gold chloride. To determine if Au is successfully deposited on the NP surface, we looked for the solution color to change from brownish to burgundy, and used other characterization methods for gold-coated NPs.

A gold surface will also help the physics by mitigating surface charge buildup due to α decay that could damage antibodies on the surface. Another possibility is that a water-soluble α emitter may not be cost-effectively disposed of if the α emitter can leach into ground water after disposal. In this case, it may be preferable to coat the rNP in a thin layer of gold. This would effectively turn them into gold NPs with an isotopic core. Chemically on the outside, they would behave like gold, but as long as the gold is sufficiently thin, (less than a few microns), the α can still radiate out and be detected. This also introduces the possibility that we can tune the energy lost in the gold, and thereby tune the energy of the α even more than just by isotope type.

Criterion 3: What is the Degradation Rate of Antibodies on the NPs when in the Presence of α Decay?

Another important criterion is how fast (if at all) Abs degrade when in the presence of the free electrons that α decay causes. Shelf-stability is important for a low-cost, widely accessible diagnostic and there is potential for the α emissions from our rNP to either break the linkage from Ab to NP surface, or to damage the Ab itself and preclude it from binding to the target biomolecule. To test this, we placed Ab-functionalized NPs (Pb, Ce, or Eu NPs) near a legally and readily available button source of Am-241, then measured two properties: i) the rate of damage to the Ab itself that affects binding to a target, and ii) the rate damage to the Ab-NP surface linkage. To measure i), we performed Ab-antigen binding assays with hCG antigen and anti-hCG on the surface of NPs comparing a control group of Ab-functionalized NPs to experimental groups of Ab-NPs that were exposed to the button source of Am-241 for up to 120 days. For ii), we measured the amount of Abs on the surface of NPs before and after a exposure using the methods described in Criterion #1.

Criterion 4: How Many rNPs are Necessary to Give Off a Detectable Signal?

Measuring the limit of detectable signals (backscatter, dead layer, energy) from a sealed Am-241 source was performed. With these data, we correlated detection limits to the other radioisotopes (Pb-210, Pu-236, Pu-238) based on their half-life and energy relative to Am-241, which informed the choice for a rNP tag.

The results of these investigations for each set of NPs guided our radioisotope selection. After selecting a suitable radioisotope, we formulated rNPs using that radioisotope and measured the binding efficiency of the rNPs with a target antigen (hCG). Mixing parameters were optimized to ensure efficient and specific binding, and the target antigen concentrations that were tested ranged from 1 aM to 1 fM (˜1-1000 molecules/μL). To perform high-accuracy investigations and test of our radio-nanoparticles detectability, strength, and binding ability, we used a lab-standard alpha spectrometer, specifically, a silicon charged-particle detector (SiCPD). These devices are similar to a simple photodiode, but of much higher quality semiconductor structure, and electrically biased with a voltage to allow amplification of the deposited energy. This electrical bias allows for much higher resolution of the exact amount of energy deposited by the alpha into the detector.

We also developed and tested various methods for detecting alpha emissions in the most sensitive and inexpensive way possible. Complementary metal-oxide-semiconductor (CMOS) sensors provide the highest confidence of success; photodiodes and α-sensitive dyes are more high risk/high reward.

For the CMOS sensor approach, we developed a prototype a detector that is capable of detecting α emissions from functionalized test strips. We used standardized a particle sources, then detected α particles emitted from rNPs developed in accordance with the above-described techniques.

We began our a detection by buying a traditional, off-the-shelf, a spectrometer. The detector consists of a silicon charged-particle detector (SiCPD), detector electronics, amplifiers, multi-channel analyzers, and analysis software. This established detector system was used to provide a comparison and control for the low-cost version of our own custom detector architecture.

SiCPDs are specifically designed to have very little dead layer (the layer on the surface detector that absorbs energy) but do not allow that energy to be measured. All detectors have some dead layer, due to electronic layers, coatings, or charge leaking. In the case of common commercially available CMOS imaging sensors, this dead layer consists of several microns of Bayer filters, to create color images, micro lenses over each pixel to increase light collection, anti-reflection coatings, and dielectric isolation layers. An α particle must pass through all these layers before they reach the charge detection layer of the p-n junction of the photodiode inside each pixel of the image sensor.

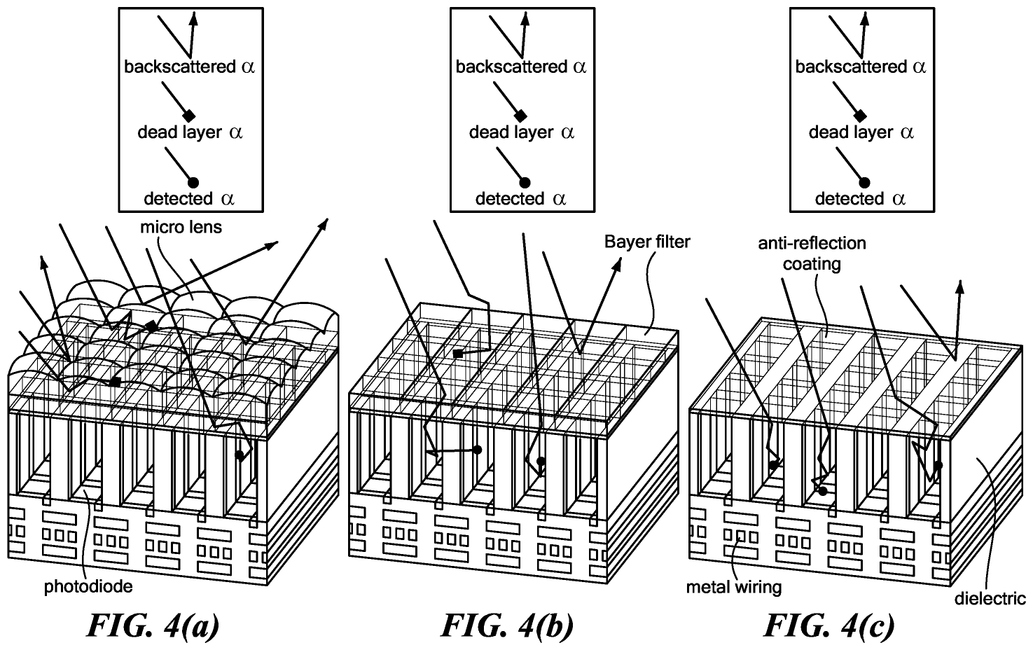

Some of these layers can be removed, such as the Bayer filter layer, and the micro lens layers, as shown in FIG. 4. This can be done mechanically or with chemical solvents, or by special OEM order in sufficient volume. Thus, FIG. 4(a) shows the off-the-shelf sensor, in which some α particles can penetrate the micro lens layers of the CMOS sensor but many are backscattered or absorbed, and not detected. FIG. 4(b) shows the sensor when the lenses are removed, and more α particles reach the Bayer filter. FIG. 4(c) shows the sensor with the Bayer filter removed, and most α particles reach the photodiode and can be detected.

Some of the dead layer cannot be removed because it is an integral component of the sensor needed to operate correctly. While all CMOS detectors have a dead layer, these data are not necessarily available from the manufacturer, we measured them ourselves on different potential sensors. This measurement was aided by modeling.

In addition to dead layer, we also measured the backscatter fraction of the front surface of each sensor we tested. Backscatter fraction is similar to dead layer, in that a particle can hit the detector but not be measured. But additionally, the particle reflects off the surface and is ejected from the detector all together before coming to a stop.

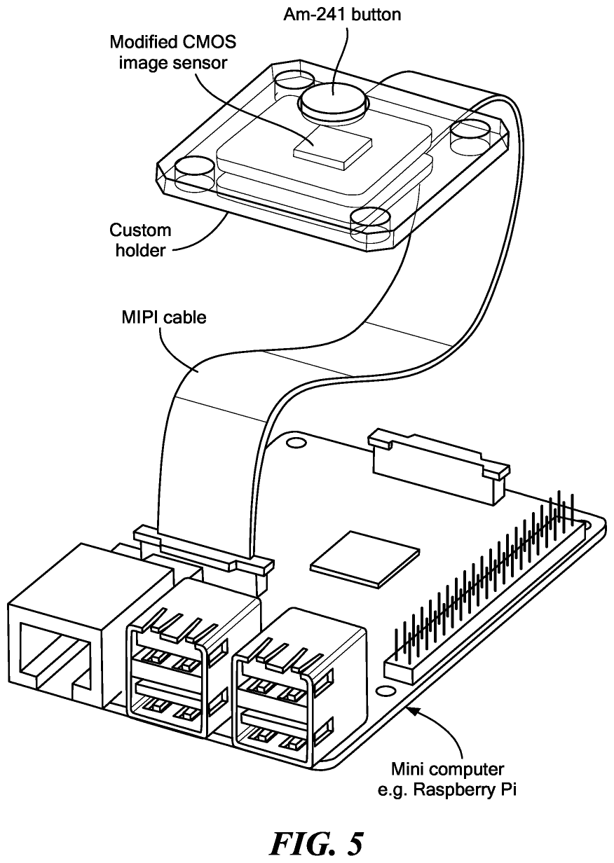

The dead layer, back scatter, and energy measurements were performed using standard methods using the test apparatus shown in FIG. 5. An α source sat on top of the modified back-illuminated CMOS sensor within a few milliliters. This distance was kept short and carefully controlled, because free alphas can be stopped even by just a few centimeters of air. The CMOS sensor was connected to a low-cost computer that directly communicated with the CMOS sensor through a mobile industry processor interface (MIPI). The mini computer allowed us to run advanced image processing code in order to identify a strike events onto the sensor. These measurements were compared to the same rate and energy measurements of a source on the SiCPD. Various thin calibration foils were inserted in-between the source and the sensor, both the prototype and the SiCPD, to make an energy spectrum comparison.

The experimental set up was modeled using Geant4. The modeling of the experimental setup involved creating a 3D computer design for the sensors, its dead layers, air, the source material and foils, and the radioisotope itself. Background radiation sources were added. We compared these models to our experimental data and adjusted appropriate unknown parameters (such as the dead layer and backscatter fraction of the various CMOS sensors) until they agreed.

Once we had a validated and calibrated model that we are confident represents out experimental setup, we used this same model paradigm to simulate α particles trapped in NPs, coated with antibodies, and surrounded by water. This modeling was an important part of identifying which radioisotope (specifically the energy of the α ejected by that radioisotope) that we selected as our potential candidate for making NPs. This allowed us to estimate the radiation dose antibodies attached to each NP will receive while they sit on the shelf.

Once we calibrated the relationship between α energy and detector response, we wrote software that can be trained on known α events and the energy expected to be measure from the event. For example, Am-241 emits three energies of alphas: 5.486 MeV (85%), 5.443 (13%) and 5.388 MeV (2%). Alphas deposited charged ions across several pixels and left distinctive marks in the form of short tracks. Simply summing up the charge collected in these tracks goes a long way to determining the energy released, but by looking at the path shape, we improved the energy deposited analysis.

We used artificial intelligence and machine learning techniques to further isolate a particles from other artifacts, to push down the limit of detection per a to as low as possible. We hope we can eventually build a system where even a single α of the right energy window is enough to detect a single bound rNP. This would enable single pathogen or biomolecule detection on the sensor. The extreme detectability of these highly energetic and localized particles allows for the exciting possibility of sub-femtomolar detection of pathogens.

We also explored alternatives to the CMOS sensor approach. It is possible that our rNP method will be so sensitive, that an ultra-low-cost test for a single pathogen will dominate over the need for the test panel capability of a CMOS sensor. In this case, it is possible to further reduce the cost of the detection device to a single photodiode and simple amplification circuit. In this case, α particles may be detected by the photo diode, with the detection concentration limited only by the background noise on the diode and detection circuit. This method works particularly well with the lateral flow strip method, because a far smaller concentration of rNPs bound to antigens can be detected than can the optical detection of Au NP using an electronic photo sensor as is used in existing lateral flow test. This is because even as few as one or two rNP can be detected due to the large number of election-hole pairs generated in a photodiode, even by a small number of alphas emitted.

Radiosensitive dyes or films may also be used. Even cheaper and simpler than the previous techniques, dyes or films may be used in a lateral flow strip-type test, but where a radioisotope sensitive dye reacts to alpha particle emission in order to change appearance such as color. These dyes can be sensitive enough to allow only a few alpha emissions over time that enable a human eye to detect visually. The advantage of such a system is that no electronics are needed, further reducing the price and device complexity. Some situations may require both extra shelf-life (for remote and difficult to resupply areas) and very weak radioactive sources (in that they produce very few alphas per minute) for either NRC approval of for binding agents that are particularly sensitive to radiological damage. Importantly, this method allows for long exposure detection of alphas, thus lengthening the shelf-life of a disposable device. Very long exposure wait times, though likely possible with a more complex and expensive detector system as described in connection with CMOS sensors, is likely not as plausible with the simpler single-diode as described just above. This is because, as pointed out earlier, the background noise becomes a low-count rate threshold for such a detector. Background noise (such as amplifier noise or thermally generated dark noise) will eventually produce some false positive detection singles; thus, radiosensitive dyes are the best option for long shelf-life testing applications.

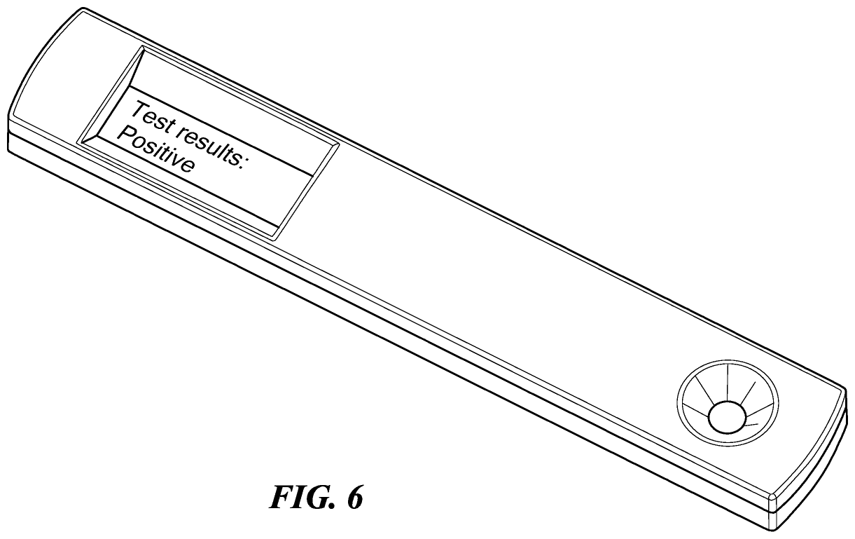

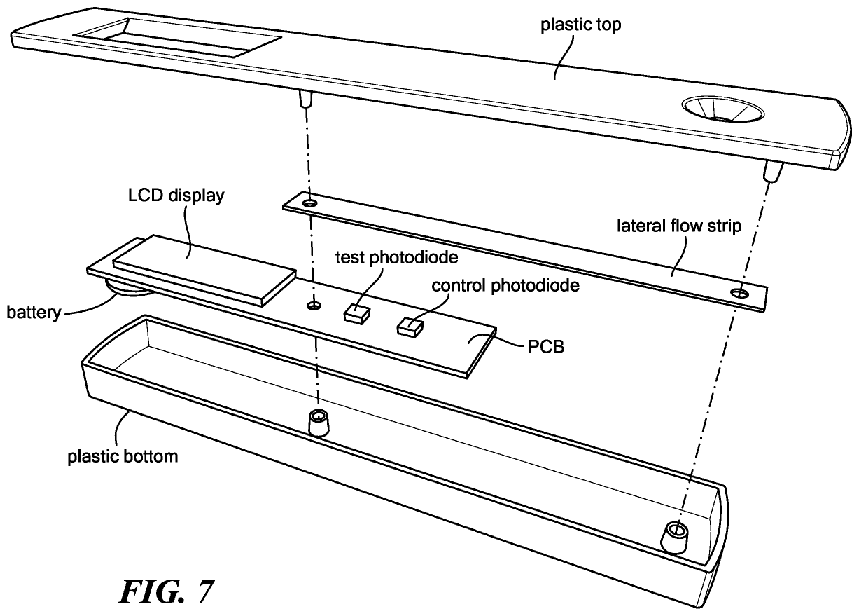

We further combined rNPs and an α-emission detection method into an integrated low-cost device that can detect target biomolecules. FIG. 6 shows an exterior view of a lateral flow device in accordance with a third embodiment of the concepts, techniques, and structures disclosed herein, while FIG. 7 depicts the hardware/electronics implementation in an exploded view.

The device's initial intended use will be for a swab sample, but other samples (saliva, blood, urine, etc.) can be accommodated in the future. For developing the prototype, we used liquid media with varying concentrations of hCG antigen (1 aM to 1 fM) and introduced the sample into the hole shown to the right of the device. Upon entering the device, the sample contacted the lateral flow strip, took up lyophilized rNPs, mixed with rNPs for rNP-target binding to occur, and a sandwich immunoassay was formed at the test volume (i.e. above the test photodiode shown in FIG. 7). A sandwich immunoassay with the antibodies from rNPs and control capture antibodies was also formed at the control line (i.e. above the control photodiode shown in FIG. 7), while the excess sample-rNP mixture continued flowing into the waste absorbent pad. When no target antigen was present, all the rNPs (besides those bound to the control line) bypassed the test line and ended up in the waste absorbent pad.

The lateral flow test strip works as follows. Once the test and control areas are bound to rNPs, they will eventually emit an alpha. This alpha will diffuse through the strip material, both losing energy and changing direction, by scattering electrons as it travels. If the alpha manages to escape the strip, it will travel through air, until it hits a photosensitive diode. In a diode, the alpha will begin again to liberate electrons and scatter off of them, creating electron-hole pairs. These electron-hole pairs are like how a photodiode detects visible photons, except that each alpha can generate hundreds of thousands, up to millions of pairs, instead of just one. Even with only one alpha entering the diode's sensitive area, is can create a detectable current pulse. Some energy may be lost to inactive areas, such as the dead layer of the diode or fibers in the strip. Each event is registered by a simple one-chip amplifier and analog-to-digital conversion (ADC) circuit attached to the diode on a low-cost printed circuit board (PCB).

Five minutes after introducing the sample to the device, a result will show up on the LCD screen showing whether the target molecule was (POSITIVE) or was not (NEGATIVE) detected. The user then places the device in a polypropylene ziploc bag and throws it away (polypropylene blocks alphas). It is important to note the rNPs are always contained within the device, with two layers of plastic in between the rNPs and the user to prevent any risk of alpha radiation contacting the user. The third layer of plastic when disposing is a final prevention measure to protect the device in the trash. The plastic disposal bag is completely optional and we expect significant noncompliance; it is the 3^{rd} redundant layer of plastic protecting the user from alpha radiation, with 2 layers in the lateral flow device.

With simple and inexpensive electronics (photodiode, small PCB), we aim to be able to make this device at scale inexpensively. This should be possible as digital pregnancy tests are currently sold at MSRP for greater cost, and we have similar electronics and materials in our lateral flow test.

It may turn out that CMOS sensors and CPU integration is required for the appropriate level of alpha detection needed. In this case, a point-of-care (POC) instrument with disposable cartridge may be used. An added benefit of a POC system rather than home use is that it can be placed in a physician's office or medical location and thereby have a lower likelihood of meeting regulatory obstacles.

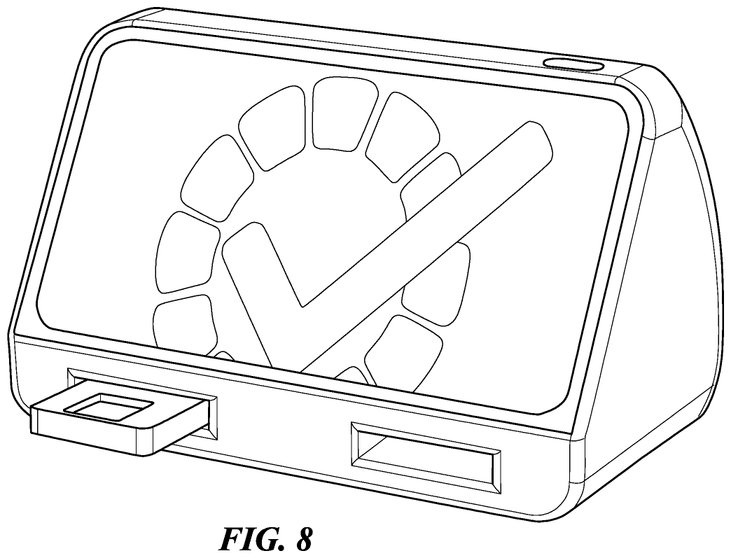

Thus, FIG. 8 shows an illustrative POC device with cartridge inserted, in accordance with a fourth embodiment of the concepts, techniques, and structures disclosed herein. We developed this POC device to be a small (<1 ft³) instrument that can be plugged into the wall of a doctor's office or battery-powered for remote use, along with a disposable cartridge. We developed this second device for two reasons. First, it will enable us to leverage the sensitive rNP detection for multiplexed detection of many biomarkers at once, which will reduce cost and waste on a per unit basis, as well as enable differentiation of pathogens in a single test (e.g. SARS-COV-2 vs. Influenza A vs. Influenza B vs. M. pneumoniae). Second, it can be a suitable alternative to the lateral flow device, as alpha particles are stopped so quickly by even the smallest amount of light weight shielding.

While traditional lateral flow paper is preferable for ultra-low cost and extensive experience of use in the diagnostic field, it may be that rNP tend to embed themselves too far into the depths of the fibers of the strip itself. This would have the effect of masking their tracks in that the alphas could lose some or all of their detectable kinetic energy in paper before reaching a detector. This could potentially limit the sensitivity of later flow style detectors. Plenty of mitigation strategies exist, such as using ultra-low-density paper, using ultra-thin lateral flow strips, higher energy alphas, or higher concentration sensitivity. However, the CMOS sensor and cartridge type-system shown in FIG. 8, where detection of alpha particles occurs directly on a prepared surface or through a micron scale window, does not have these shielding issues.

As has been shown already, modified CMOS detectors are sensitive enough to identify single alphas, both per pixel, and using image processing and machine learning techniques for multi-pixel spectral analysis. They also have megapixel resolution at little cost. This large pixel screen allows for many sub-regions to be reserved geometrically for multiplexing several (at least hundreds or even thousands) of diagnostic regions all with one single mega pixel measurement. Standard image capture, on a specialized CMOS imager using a low-cost computer, can test many different rNP regions at once, with each region containing capture antibodies that bind to different antigens or pathogens, at no additional cost or complexity added to the detector system. The only lower limit to the number of simultaneous diagnostic regions is the drift region of an active alpha, which in silicon is only a few tens of microns, thus guiding the appropriate buffering between regions.

For example, even the smallest commercially available ¼″ CMOS imager, divided into 100 μm×100 μm sub-diagnostic regions could test for as many as two thousand different target biomolecules. In the largest of detectors, full frame CMOS imagers, though more expensive, could yield as many as sixty thousand separately sensitive test areas. This not only allows for the astonishing possibility of a very large number of low-cost diagnostics being performed on a single test sample, but also plenty of room for calibration, control and redundant systematic check vectors.

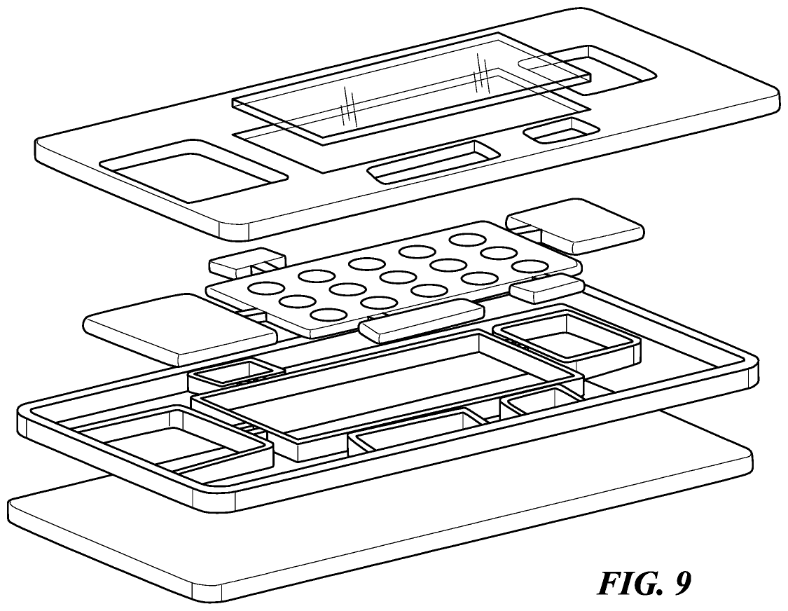

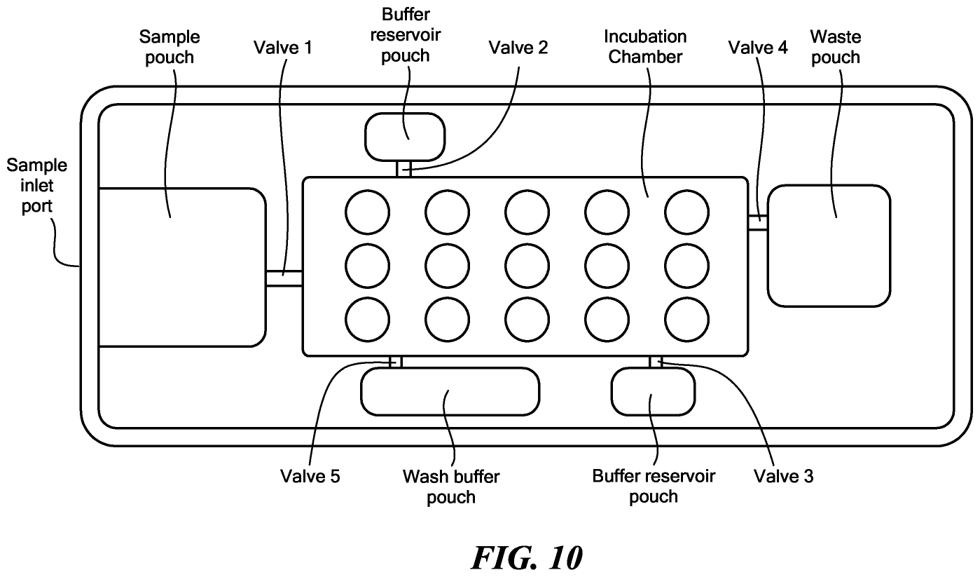

As a demonstration, we tested our POC device and cartridge's ability to detect 15 targets simultaneously. FIG. 9 shows an exploded view of a cartridge suitable for insertion in the POC device, while FIG. 10 shows a top view of the cartridge with relevant components labeled. The cartridge can be made with 3D printing or other standard lithography techniques as necessary.

The POC device/cartridge system works as follows. The sample swab is inserted into the sample inlet port and rotated back and forth for approximately 10 seconds to ensure sample mixing in the buffer in the sample pouch. Then the swab is removed, the sample port is capped, and the cartridge is inserted into the instrument as shown in FIG. 8.

All valves are opened and closed by instrument pistons applying or removing pressure on the flexible channels or pouches. Valve 1 is opened, and the content of the sample pouch is transferred to the incubation chamber through palpitation by a piston, after which Valve 1 is closed.

Valves 2 and 3 are then opened and the mixing occurs in the incubation chamber through agitation caused by repeated alternate palpitation (5× on each buffer chamber) of buffer reservoir pouches. Valves 2 and 3 are closed.

The mixture is allowed to sit in the incubation chamber for an appropriate duration (such as 20 minutes) to allow for binding reactions to occur of the target biomolecule to the capture antibodies spotted on each circle in FIG. 10. In some embodiments, each circle contains capture molecules for binding to different target molecules. In this way, a single cartridge may detect the presence of multiple target molecules in a single sample.

After incubation, Valve 4 is opened and the waste is aspirated into the waste chamber. Valve 4 is closed.

Valve 5 is opened, and a wash buffer is transferred from the wash buffer pouch into the incubation chamber through palpitation by a piston. Valve 5 is closed.

Valve 4 is opened, and the waste is aspirated into the waste chamber. Valve 4 is closed.

Once the assay processes conclude, the CMOS detector clamps flush to the top of the incubation chamber and readings are recorded through the mylar window (translucent rectangle at the top of FIG. 9). The POC device may then display the results on a digital display.

It is to be understood that the phraseology and terminology employed herein are for the purpose of description and should not be regarded as limiting. As such, those skilled in the art will appreciate that the conception, upon which this disclosure is based, may readily be utilized as a basis for the designing of other structures, methods, and systems for carrying out the several purposes of the disclosed subject matter.

In the foregoing detailed description, various features of embodiments are grouped together in one or more individual embodiments for the purpose of streamlining the disclosure. This method of disclosure is not to be interpreted as reflecting an intention that the claims require more features than are expressly recited therein. Rather, inventive aspects may lie in less than all features of each disclosed embodiment.

Although the disclosed subject matter has been described and illustrated in the foregoing exemplary embodiments, it is understood that the present disclosure has been made only by way of example, and that numerous changes in the details of implementation of the disclosed subject matter may be made without departing from the spirit and scope of the disclosed subject matter.

As used herein, “including” means including without limitation. As used herein, the terms “a” and “an”, when modifying a noun, do not imply that only one of the noun exists. As used herein, unless the context clearly indicates otherwise, “or” means and/or. For example, A or B is true if A is true, or B is true, or both A and B are true. As used herein, “for example”, “for instance”, “e.g.”, and “such as” refer to non-limiting examples that are not exclusive examples. The word “consists” (and variants thereof) are to be give the same meaning as the word “comprises” or “includes” (or variants thereof).

Various embodiments of the concepts, systems, devices, structures and techniques sought to be protected are described herein with reference to the related drawings. Alternative embodiments can be devised without departing from the scope of the concepts, systems, devices, structures and techniques described herein. It is noted that various connections and positional relationships (e.g., over, below, adjacent, etc.) are set forth between elements in the following description and in the drawings. These connections and/or positional relationships, unless specified otherwise, can be direct or indirect, and the described concepts, systems, devices, structures and techniques are not intended to be limiting in this respect. Accordingly, a coupling of entities can refer to either a direct or an indirect coupling, and a positional relationship between entities can be a direct or indirect positional relationship.

As an example of an indirect positional relationship, references in the present description to forming layer “A” over layer “B” include situations in which one or more intermediate layers (e.g., layer “C”) is between layer “A” and layer “B” as long as the relevant characteristics and functionalities of layer “A” and layer “B” are not substantially changed by the intermediate layer(s). The following definitions and abbreviations are to be used for the interpretation of the specification. As used herein, the terms “comprises,” “comprising, “includes,” “including,” “has,” “having,” “contains” or “containing,” or any other variation thereof, are intended to cover a non-exclusive inclusion. For example, a composition, a mixture, process, method, article, or apparatus that comprises a list of elements is not necessarily limited to only those elements but can include other elements not expressly listed or inherent to such composition, mixture, process, method, article, or apparatus.

Additionally, the term “exemplary” is used herein to mean “serving as an example, instance, or illustration. Any embodiment or design described herein as “exemplary” is not necessarily to be construed as preferred or advantageous over other embodiments or designs. The terms “one or more” and “one or more” are understood to include any integer number greater than or equal to one, i.e. one, two, three, four, etc. The terms “a plurality” are understood to include any integer number greater than or equal to two, i.e. two, three, four, five, etc. The term “connection” can include an indirect “connection” and a direct “connection.”

References in the specification to “one embodiment, “an embodiment,” “an example embodiment,” etc., indicate that the embodiment described can include a particular feature, structure, or characteristic, but every embodiment can include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

For purposes of the description hereinafter, the terms “upper,” “lower,” “right,” “left,” “vertical,” “horizontal, “top,” “bottom,” and derivatives thereof shall relate to the described structures and methods, as oriented in the drawing figures. The terms “overlying,” “atop,” “on top, “positioned on” or “positioned atop” mean that a first element, such as a first structure, is present on a second element, such as a second structure, where intervening elements such as an interface structure can be present between the first element and the second element. The term “direct contact” means that a first element, such as a first structure, and a second element, such as a second structure, are connected without any intermediary elements.

Use of ordinal terms such as “first,” “second,” “third,” etc., in the specification to modify an element does not by itself connote any priority, precedence, or order of one element over another or the temporal order in which acts of a method are performed, but are used merely as labels to distinguish one element having a certain name from another element having a same name (but for use of the ordinal term) to distinguish the elements.

The terms “approximately” and “about” may be used to mean within ±20% of a target value in some embodiments, within ±10% of a target value in some embodiments, within ±5% of a target value in some embodiments, and yet within ±2% of a target value in some embodiments. The terms “approximately” and “about” may include the target value. The term “substantially equal” may be used to refer to values that are within ±20% of one another in some embodiments, within ±10% of one another in some embodiments, within ±5% of one another in some embodiments, and yet within ±2% of one another in some embodiments.

The term “substantially” may be used to refer to values that are within ±20% of a comparative measure in some embodiments, within ±10% in some embodiments, within ±5% in some embodiments, and yet within ±2% in some embodiments. For example, a first direction that is “substantially” perpendicular to a second direction may refer to a first direction that is within ±20% of making a 90° angle with the second direction in some embodiments, within ±10% of making a 90° angle with the second direction in some embodiments, within ±5% of making a 90° angle with the second direction in some embodiments, and yet within ±2% of making a 90° angle with the second direction in some embodiments.

What may be claimed includes:

1. A test device for detecting the presence of a target molecule in a liquid sample, the test device comprising:
a permeable material defining at least a first portion and a second portion, the portions being in the same plane so as to permit capillary flow communication with each other;
the first portion for receiving the liquid sample, the first portion comprising a plurality of molecular tags, each molecular tag comprising an α-emitter coupled to capture molecules for binding to the target molecule in its presence; and
the second portion for detecting presence of the target molecule, the second portion comprising a testing volume having capture molecules for binding to the target molecule in its presence; and
an α-particle detector for detecting α particles emitted from the testing volume.

2. The test device according to claim 1, wherein the target molecule comprises a pathogen, or an antigen, or deoxyribonucleic acid (DNA), or ribonucleic acid (RNA), or a toxin.

3. The test device according to claim 1, wherein at least one of the capture molecules comprises an antibody, or a nucleic acid, or an aptamer.

4. The test device according to claim 1, wherein the α-emitter comprises a radioactive nanoparticle (rNP).

5. The test device according to claim 4, wherein the rNP is coated with an environmental protectant.

6. The test device according to claim 1, wherein the α-emitter comprises polonium-210.

7. The test device according to claim 1, wherein the α-emitter has a half-life between 1 day and 2 years.

8. The test device according to claim 1, wherein the α-emitter decays to a stable isotope with a probability of greater than 90%.

9. The test device according to claim 1, wherein the α-emitter has a branching ratio of below 10% with respect to β and γ decays.

10. The test device according to claim 1, wherein the α-emitter has decay products that have a branching ratio of below 10% with respect to β and γ decays.

11. The test device according to claim 1, wherein the α-particle detector comprises an array of complementary metal-oxide-semiconductor (CMOS) diodes or a charge-coupled device (CCD).

12. The test device according to claim 1, further comprising an indicator that indicates when the α-particle detector has detected α particles emitted from the testing volume.

13. The test device according to claim 1, wherein:
the first portion further comprises a plurality of second molecular tags for binding to non-target molecules in the liquid sample;
the second portion further comprises a control volume having second capture molecules for binding to the non-target molecules; and
the test device further comprises a second detector for detecting the presence of second molecular tags within the control volume.

14. The test device according to claim 13, further comprising an indicator that indicates when the second detector has detected the presence of second molecular tags within the control volume.

15. An assay for detecting the presence of a target molecule in a liquid sample, the assay comprising:
receiving the liquid sample;
mixing the received liquid sample with a plurality of molecular tags to form a mixed sample, each molecular tag comprising an α-emitter coupled to capture molecules for binding to the target molecule in its presence;
allowing the mixed sample to flow to a testing volume, the testing volume having capture molecules for binding to the target molecule in its presence; and
using an α-particle detector to detect α particles emitted from the testing volume.

16. The assay according to claim 15, wherein the target molecule comprises a pathogen, or an antigen, or deoxyribonucleic acid (DNA), or ribonucleic acid (RNA), or a toxin.

17. The assay according to claim 15, wherein at least one of the capture molecules comprises an antibody, or a nucleic acid, or an aptamer.

18. The assay according to claim 15, wherein the α-emitter comprises a radioactive nanoparticle (rNP).

19. The assay according to claim 18, wherein the rNP is coated with an environmental protectant.

20. The assay according to claim 15, wherein the α-emitter comprises polonium-210.

21. The assay according to claim 15, wherein the α-particle detector comprises an array of complementary metal-oxide-semiconductor (CMOS) diodes or a charge-coupled device (CCD).

22. The assay according to claim 15, further comprising providing a perceptible indication when the α-particle detector has detected α particles emitted from the testing volume.

23. The assay according to claim 15, wherein the permeable material has a control volume with second capture molecules for binding to non-target molecules in the liquid sample, the assay further comprising:
mixing the received liquid sample with a plurality of second molecular tags for binding to non-target molecules in the liquid sample;
allowing the mixed sample to flow to the control volume; and
using a second detector for detecting the presence of second molecular tags within the control volume.

24. The assay according to claim 23, further comprising providing a perceptible indication when the second detector has detected the presence of second molecular tags within the control volume.
